(19) United States
(12) Patent Application Publication
(10) Pub. No.: US 2018/0318544 A1
(43) Pub. Date: Nov. 8, 2018

(54) BRAINWAVE ENTRAINMENT METHOD ALTERING SPECIFIC PARTS OF THE BRAIN

(71) Applicant: Ugur Dogan, Yogyakarta (ID)

(72) Inventor: Ugur Dogan, Yogyakarta (ID)

(21) Appl. No.: 15/585,313

(22) Filed: May 3, 2017

(51) Int. Cl. A61M 21/02

(52) U.S. Cl. CPC A61M 21/02; A61M 2021/0027

(57) ABSTRACT

A method for incorporating brain wave entrainment into an audio composition by selectively modulating sound elements within the composition. The invention provides a way to specify and modulate individual frequency components in an audio composition, according to alter and stimulate the desired parts and areas of the brain or the body.

### BACKGROUND

The electrical activity in the brain produces detectable signals known as brain waves, the frequency of which is associated with changes in mental state and cognitive ability. During daily waking life, a normal human brain functions at frequencies ranging from 13 to 25 Hertz (Hz), or cycles per second, called the beta state. During times of rest or daydreaming, brain wave cycles drop to 8 to 12 cycles per second, or the Alpha state. When sleeping, Theta and Delta states are dominant, ranging from 4 to 7 and 0.2 to 4 cycles per second respectively.

Brain waves can be altered if a person is presented with precisely timed, repetitive stimuli through the ears or eyes at repetition rates equal to the desired brain wave frequency. This is called brain wave entrainment. Because brain waves relate to mood and attention levels, researchers believe that stimulating frequencies can assist in a variety of activities, including relaxation, concentration, sleep and more.

However all brain wave entrainment inventions and methods are only “ whole brain” measurements and solutions. The measured electrical activity reflects both the intrinsic activity of neurons in the cerebral cortex and the information sent to it by subcortical structures and the sense receptors.

This composite activity is called an electroencephalogram or EEG. An EEG electrode will mainly detect the activity in the brain region just under it. The electrodes receive the activity from only thousands of neurons. In fact, one square millimeter of cortex has more than 100,000 neurons. It is therefore far too simplistic and too limited method to measure the activity of all the brain and far too limited to find answers.

A brain wave entrainment method to alter specific parts and areas of the brain have a far more reach of measurement to find solutions and enhance brain functions or treat brain disorders.

### BRIEF SUMMARY OF THE INVENTION

The present invention relates to a method of altering different parts and areas of the brain using brain wave sound patterns; more specifically, the invention relates to a method altering specific parts and areas of the human brain through audio compositions.

### BRIEF DESCRIPTION OF THE DRAWINGS

FIG. 1. A flow chart of a software system that may be used to separate and modulate individual frequency components according to a brain wave protocol.

FIG. 2. An example of a visual display typical to the present invention.

### DETAILED DESCRIPTION AND BEST MODE OF IMPLEMENTATION

Brain is made up of billions of brain cells called neurons, which use electricity to communicate with each other. The combination of millions of neurons sending signals at once produces an enormous amount of electrical activity in the brain, which can be detected using sensitive medical equipment (such as an EEG), measuring electricity levels over areas of the scalp.

The combination of electrical activity of the brain is commonly called a brainwave pattern, because of its cyclic, “wave-like” nature.

Attempts have been made to utilize audio signals for the purpose of directly altering a person's brainwave state. These attempts have employed the use of audio signals of differing frequencies applied to the ears of a listener. The desired result is that the listener's brain will change its own frequency to match that of the audio signal. For instance, a low frequency signal will generally cause a listener's initially excited brainwave state to decrease to a lower frequency, more relaxed state. However, a need still remains, a better method of specifically achieving brainwave states in individuals.

Stimulating specific brain parts and areas is a brand new and the better method achieving brainwave states to enhance brain functions and find more effective solutions to treat brain disorders.

It is to be understood that the descriptions below are merely illustrative of the presently preferred embodiments of the invention and that no limitations are intended to the detail of construction or design herein shown other than as defined in the appended claims.

The method both selected frequency number ranges and the used technology of descending and/or ascending sound signals and its modulations are all a part of the new method for altering brain states on specific parts of the brain. The frequency number ranges chosen or discovered are generally in correlation with Brodmann areas. A Brodmann areas are regions of the cerebral cortex, in the human or other primate brain, defined by its cytoarchitecture, or histological structure and organization of cells. In accordance with an aspect of the present invention, a method for altering a brain part of a person, includes means for producing a two or more audio signals to be supplied to both ears of the person; means for substantially varying the frequency of at least one of the first and second audio signals such that substantially varying monaural beats and isochronic tones are produced in the ear and in the brain of the person when the two or more audio signals are supplied to both the ears, respectively.

It is, therefore an object of the invention to provide different combinations of the frequency master groups ranges and slave subgroups ranges for stimulating specific parts and areas of the brain. Master groups ranges linked to brain regions and slave subgroup ranges linked to the specific parts or areas of the specified master group brain regions.

It is a further object of the invention, to provide an application for the induction of brain wave patterns associated with a part of the brain in a subject, as above, which utilizes a pleasing sound that is modulated and programmed in such manner as to induce the brain wave patterns only in those brain structures where it naturally should occur.

### Brain Wave Protocol

A brain wave protocol is used to express the desired brain wave frequencies across the timeline of an audio composition generated through a tone generator and modulator. This is represented using a chart with a time span represented horizontally and brain wave frequencies vertically.

The brain wave protocol is divided into frequency group numbers, sub group numbers and rates of modulation.

The frequency group number represents the brain region and the sub group numbers the parts of that brain region.

For example, frontal cortex has a group frequency number of XX cycles per second. Stimulating different parts of the frontal cortex are done through the sub group frequency numbers. Modulation is partly in correlation with the chosen frequency numbers.

### Software Model

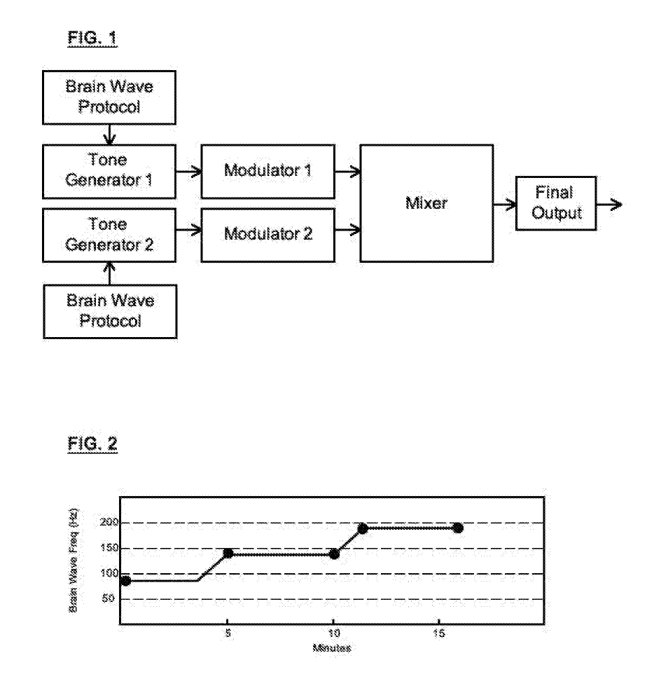

FIG. 1 depicts a logical flow chart of the computer functions to be performed in accordance with the present invention.

### Operation

FIG. 2 depicts a timeline of a session stimulating different parts of the brain.

The descriptions should not be construed as limitations on the scope of the invention, but rather as an exemplification of one preferred embodiment thereof. Many other variations are possible.

I claim:

1. A method for altering specific parts and areas of the human brain through brain wave entrainment audio compositions, comprising:
a) means of selecting an audio composition, the audio composition comprising a plurality of frequency components;
b) means of selecting a brainwave protocol, the brainwave protocol comprising the desired brain wave frequencies across a timeline of the audio composition;
c) means of modulating at least one of the plurality of signals according to the selected brainwave protocol;
d) providing specific brain wave frequency combinations means of which parts of the brain being altered;
e) providing combinations of master and slave brain wave frequency numbers means of stimulating specific areas of the brain, whereby the master numbers states the brain regions and the slave numbers the specific parts of that brain regions means in general according to Brodmann areas;
f) providing brainwave frequency modulation parameters for master and slave brain wave frequency numbers, whereby the parameters correlated with the frequency numbers;
g) providing brain wave frequency numbers in decimal number ranges means of stimulating specific brain parts and areas more in detailed form;
h) means of stimulating any parts, areas or systems of the body;
i) means of remixing the signals to produce a final digital output; and
j) means of treating or enhancing any parts or areas of the brain or the body;
